(19) United States
(12) Patent Application Publication
(10) Pub. No.: US 2011/0089260 A1
(43) Pub. Date: Apr. 21, 2011

(54) REMOTE CONTROLLED AEROSOL DISPENSING SYSTEM

(76) Inventor: John Van Roemburg, Brighton Victoria (AU)

(21) Appl. No.: 12/741,826

(22) PCT Filed: Nov. 7, 2008
(86) PCT No.: PCT/AU2008/001655

§ 371 Date: May 6, 2010

(30) Foreign Application Priority Data
Nov. 7, 2007 (AU) 2007906071

(51) Int. Cl. A62C 13/62; A24F 25/00

(52) U.S. Cl. 239/303; 239/34

(57) ABSTRACT

An apparatus for dispensing aerosols includes a plurality of aerosol dispensers with each aerosol dispenser associated with a reservoir of liquid for dispersion and being actuated and controlled by a single control unit. Preferably, scented oils are dispersed by the apparatus and, while a wide variety of dispersion devices may be used, a piezo-electrically activated dispersion is preferred.

### AREA OF THE INVENTION

This invention relates to the area of scenting equipment for dispensing fragrance into the air, or into water. In particular the invention relates to an atomizing apparatus used in connection with a fluid reservoir and to methods of atomizing such fragrances including means whereby a plurality of atomizers can be remotely controlled from a central location.

### BACKGROUND TO THE INVENTION

The dispersion of scents in air has become a popular method of eliminating unwanted odours in the ambient environment and also is used for marketing purposes to encourage shoppers to purchase food stuffs or to otherwise experience a pleasant shopping environment.

Essential oils are also dispersed in this manner to oppose air borne microbes or bacteria, viruses and moulds among other things as well as promoting a healthy pleasant and positive environment.

It is known for such scenting equipment to have control means which are typically fitted on or near the scenting device. It is also known for wireless remote control of single units to exist.

Scenting equipment to date does not however have centralized control at a large distance from the scenting device or devices and is not adapted to control a network of such scenting points.

### OUTLINE OF THE INVENTION

It is an object of the invention to provide a means whereby the output of a multiplicity of air scenting devices can be controlled either individually or simultaneously by a single control means.

It is a further object of the invention that an improved means of actuating dispersion from a scenting device be provided in association with such a system.

The invention in a first aspect provides a system for dispensing aerosols which includes a plurality of remote aerosol dispensers actuated and controlled by a single control unit.

In the invention the term aerosol dispenser includes any type of nebulizer or atomiser.

The invention in a second aspect is an aerosol dispenser for use with this system which dispenser includes a reservoir for the fluid to be dispersed and piezoelectric means for actuating such dispersion.

It is preferred that the aerosol dispensing system control unit be able to communicate information to the remote dispensers, such information including scenting strength, choice of fluid to be dispersed, duty cycle, scheduling, signalling and the like.

It is further preferred that information be fed back to the control unit from the remote dispensers, such information including parameters such as environmental parameters such as ambient temperature, humidity, presence of people in the vicinity of the dispenser, the state of the dispenser and its fluid level, fluid type and functionality of the system. Any preferred information may be included.

It may further be preferred that a remote dispenser have an operation panel which can be used to transmit manual inputs back to the control unit within limits set by the control unit.

It may also be preferred that the communication between the control unit and the remote dispensers be effected by radio means although any preferred means may be used.

In the event that dispensers are located out of range of the direct control unit to dispenser distance it is preferred that a wireless system with a hopping or rerouting facility be provided to communicate to the distant dispensers.

It is preferred in the invention that the control system have the capability to monitor the sensory feedback from the environment of a dispenser, make computations and if desired, either by operator or automatically, adjust parameters such as operating schedule, choice of liquid and strength of dispersion of the aerosol.

It is further preferred that the control system have the capacity to monitor dispenser status details such as the consumption of the liquid and also generate reports, signal the need for replenishment or analyse the effectiveness of the aerosol diffusion as perceived by humans.

While any fluid may be dispersed with the system of the invention it is preferred that scented oils be used.

It is preferred that each dispenser diffuse oil into the air at a chosen rate and that each dispenser have a reservoir for storing the oil.

While any type of nebulizer or atomiser or aerosol dispenser of any kind may be used in the invention it is preferred that a piezo-electrically operated dispenser be used.

It is further preferred that the liquid flow to the dispenser through an opening of the reservoir, possibly aided by a tube, mesh or a wick.

It may further be preferred that the amount of oil supplied to the dispenser be regulated by means of a ‘water lock’.

It may also be preferred that the dispenser be integrated and sealed with the reservoir, for a replaceable unit. An electric interface allows easy mounting into the housing when replacing the unit.

It is further preferred that the dispenser used have a reservoir for the scented oil which is associated with means whereby the oil is drawn from the reservoir to a dispersion area by wick means where it is dispersed by piezoelectric means.

Alternative means of aerosol release include evaporation by natural or forced convection or heating and/or vibration.

In order that the invention may be more readily understood we will describe by way of non limiting example a preferred embodiment of the invention.

### BRIEF DESCRIPTION OF THE DRAWING FIGURES

FIG. 1 Shows a schematic diagram of the aerosol dispensing system of the invention;

FIG. 2 Shows a piezo electric integrated cartridge with an overlying oil reservoir;

FIG. 3 Is a perspective view of the outside of the cartridge of FIG. 2;

FIG. 4 Shows an alternate integrated piezo actuated oil cartridge;

FIG. 5 Shows a diagram of a multiple scenting device;

FIG. 6 Shows a diagram of an integrated oil cartridge in which the oil to be dispersed is soaked in a liquid absorbent material such as a sponge;

### DESCRIPTION OF AN EMBODIMENT OF THE INVENTION

### The System of the Invention

The invention provides a system for dispensing aerosols which includes a plurality of remote aerosol dispensers actuated and controlled by a single control unit.

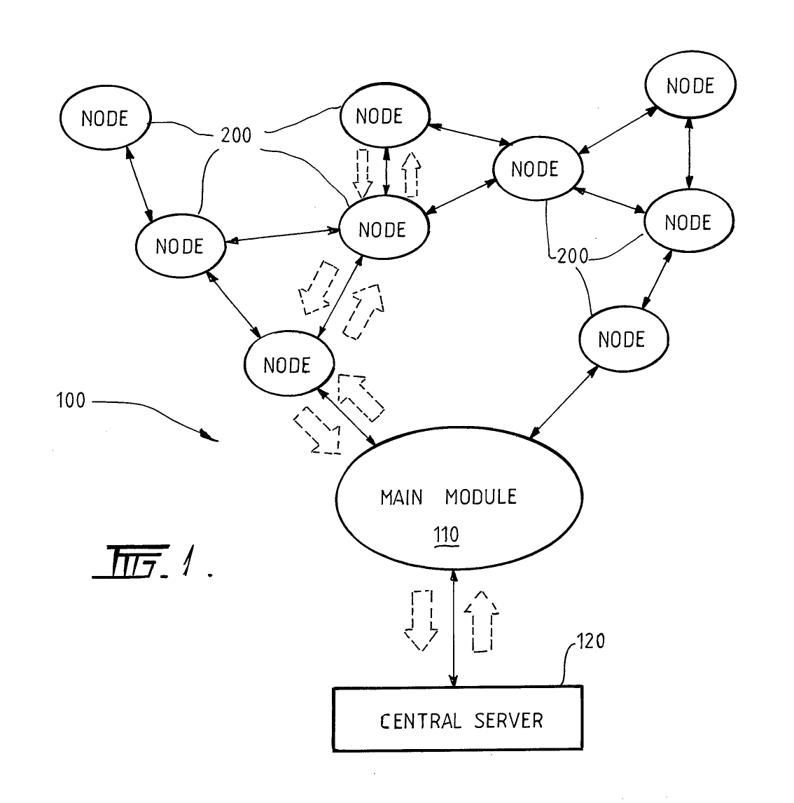

FIG. 1 is a schematic diagram of the communication system between the single control unit 100, which includes a main module 110 and central server 120, and the remote nodes or dispensers 200.

The central server can communicate with all the nodes in the system either directly or via other nodes in the system. The arrangement is such that if one route to a node is interrupted because a node fails to communicate automatic rerouting is provided. In addition unique node identification and encryption is provided such that only the intended node can receive the relevant information.

While any type of communication may be used in the invention between the main module and remote dispensers it is preferred that radio communication be used.

The preferred aerosol dispenser for use with this system is one having a reservoir for the fluid to be dispersed and piezoelectric means for actuating such dispersion however the specific type used is not restricted in the invention and atomizing apparatus or dispensers may consist of nebulizers, atomizers driven by air or by piezo means.

The type of liquids used with this type of apparatus typically include aromatic or essential oils, either pure or mixed with water. While the invention relates to a means of dispersing all manner of fluids for convenience sake here the invention will be discussed in terms of its application to the dispersion of scented oils.

Dispensers typically have a reservoir for storing liquids and may consist of a diffuser combined with the reservoir to form an integrated, interchangeable cartridge. The preferred cartridge of the invention has a mechanical and electrical interface to the housing of the scenting point.

The system of the invention includes multiple dispensers (scenting points) which can be controlled and programmed as a group or individually from a central location and the system is mains powered with battery back up.

In case the central control unit is absent or non-operational, the scenting points can work independently being controlled and programmed on the unit, while the central system can retake control at any point in time. Scenting points can communicate and function even when disconnected from the mains input power. Sleep mode operation allows for low battery consumption.

The system can have multiple routing capabilities, signal encryption and time-out capabilities. A wireless network with re-routing nodes allows the network to be larger than the range of a point to point wireless communication. The scenting points can execute their scheduled program, even when disconnected from the network, or from the main power input.

Programming of scenting points allows setting the choice of scent, the strength or duty cycle, and scheduling according to preference, or based on input sensors in the environment near the scenting point, and of the scenting point itself. Sensory feedback of the scenting point and environment can be communicated back to the control system, using a wired or wireless network.

While any communication means may be used in the invention it is preferred that the communication between the control unit and the remote dispensers is effected by radio means. In the event that dispensers are located out of range of direct control unit to dispenser distance it is preferred that a wireless system with a hopping or rerouting facility be provided to communicate to the distant dispensers.

In the invention a graphical user interface is provided which is the communication tool between the main module 110 and the remote dispensers 200 and is used for communicating searches, reports and the like and commands to remote scenting devices (dispensers).

Specific software is provided which enables an operator to communicate between a computer and wired and wireless dispensers. This software displays the detection of multiple scenting devices and settings for such devices.

Options available include an option for searching scenting devices or settings of such devices through a built in search tool and determining status parameters of these devices such as cartridge volumes, types of scents etc.

The interface can work within the boundaries of a local area network (LAN) or using the Internet to access the control unit or computer remotely. The interface operates on a search tool which will assist with allocating scent, floors, spaces (room identification) or areas, scenting devices, IP addresses and serial numbers.

The system of the invention also permits environmental information be fed back to the control unit from the remote dispensers or scenting points, such information including parameters such as ambient temperature, humidity, presence of people in the vicinity of the dispenser, the state of the dispenser and its fluid level. Any preferred information may be included.

By this means a system of multiple dispensing apparatus (scenting points) can be controlled and programmed as a group or individually from a central location. In case the central control is absent or non-operational, the scenting points can work independently, being controlled and programmed on the unit, while the central system can retake control at any point in time. Scenting points can communicate and function even when disconnected from the mains input power. Sleep mode operation allows for low battery consumption.

The system can have multiple routing capabilities, signal encryption and time-out capabilities. A wireless network with re-routing nodes allows the network to be larger than the range of a point to point wireless communication. The scenting points can execute their scheduled program, even when disconnected from the network, or from the main power input.

Programming of scenting points allows setting the choice of scent, the strength or duty cycle, and scheduling according to preference, or based on input sensors in the environment near the scenting point, and of the scenting point itself. Sensory feedback of the scenting point and environment can be communicated back to the control system, using a wired or wireless network.

### A Preferred Dispenser

As previously mentioned a preferred dispenser for use at a scenting point would include a reservoir of scented oil which could be provided with a wick device passing from an upper dispersion surface into the reservoir such that a constant supply of oil to be dispersed was available at the dispersion surface.

Many means of dispersing the oil may be used however the simple passage of air over this source could be used or alternatively piezo electric means can be used to disperse this oil to the atmosphere.

In the invention it is preferred that piezo electric means be used to disperse aerosols of oils although a variety of dispensers may be used as described here.

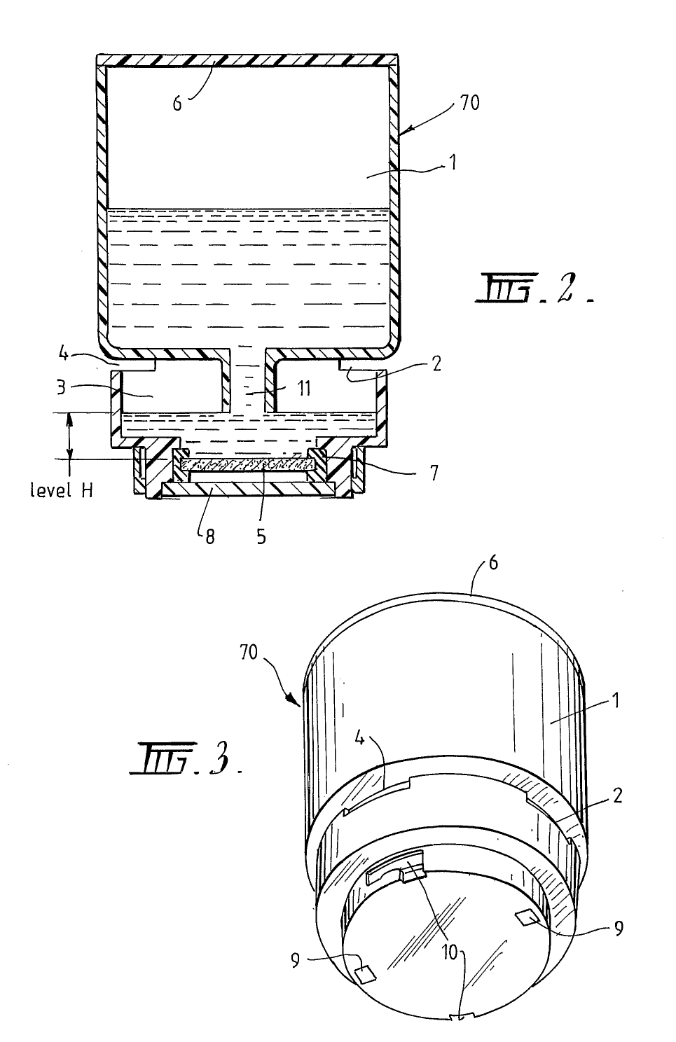

FIGS. 2 and 3 show one embodiment of a piezo electrically actuated dispenser integrated with a reservoir into a single cartridge 70.

The sealed reservoir with built in atomiser allows optimisation of atomizing parameters to the actual properties of the liquid contents of the reservoir. Parameters can be the dimension of the various parts such as hole diameters and length and gaps.

The nature of the liquids that are diffused tend to affect the material of the atomiser giving it a maximum operating time. A disposable cartridge has the advantage that it can be made at low cost and uses less material than atomisers that require repeated use.

The cartridge of the invention 70 includes a reservoir 1 in which the liquid to be dispersed is stored. A lid 6 closes the reservoir so that the liquid can only exit through outlet 11 to supply liquid to the diffusion chamber 3. The piezo element 5 then excites the liquid and creates a mist.

Rubber part 7 prevents liquid leakage from the diffusion chamber 3 and holds the piezo assembly which is locked by cover 8. The mist then passes from the diffusion chamber in air passing through air inlet 4 and outlet 2.

The unit is enabled for use as a cartridge by means of electrical interface 9 and mechanical interface 10.

The separation of the reservoir 1 and the diffusion chamber 3 is designed to create a constant level of liquid H above the piezo activator. This principle is also known as the bird feed system.

Common piezo diffusion systems use water based liquids that do not require that a precise height H be maintained for mist production to occur. For liquids such as essential oils the allowable height variation is less and a means of controlling the level of liquid above the piezo element is required.

The separation of the liquid in the reservoir from the diffusion chamber plus the feeding of the liquid as it is being used up results in amore constant and optimum diffusion rate. It also allows a longer period of time to replenish the liquid because the size of the reservoir can be larger than the normal level H variation allows.

Another embodiment of a piezo electrically actuated oil cartridge is shown in FIG. 4. In this embodiment the oil to be dispersed is stored in a reservoir 38 while the piezo 31 diffuses the liquid from the piezo holder 32. A seal 33 prevents leaking and a wick 34 replenished the liquid from the bottle 38.

A wick holder 35 positions the wick at the correct distance to the piezo and on down to the bottom of reservoir 38.

A single or double part cap 36, 37 seals the cartridge to both prevent leakage and refilling.

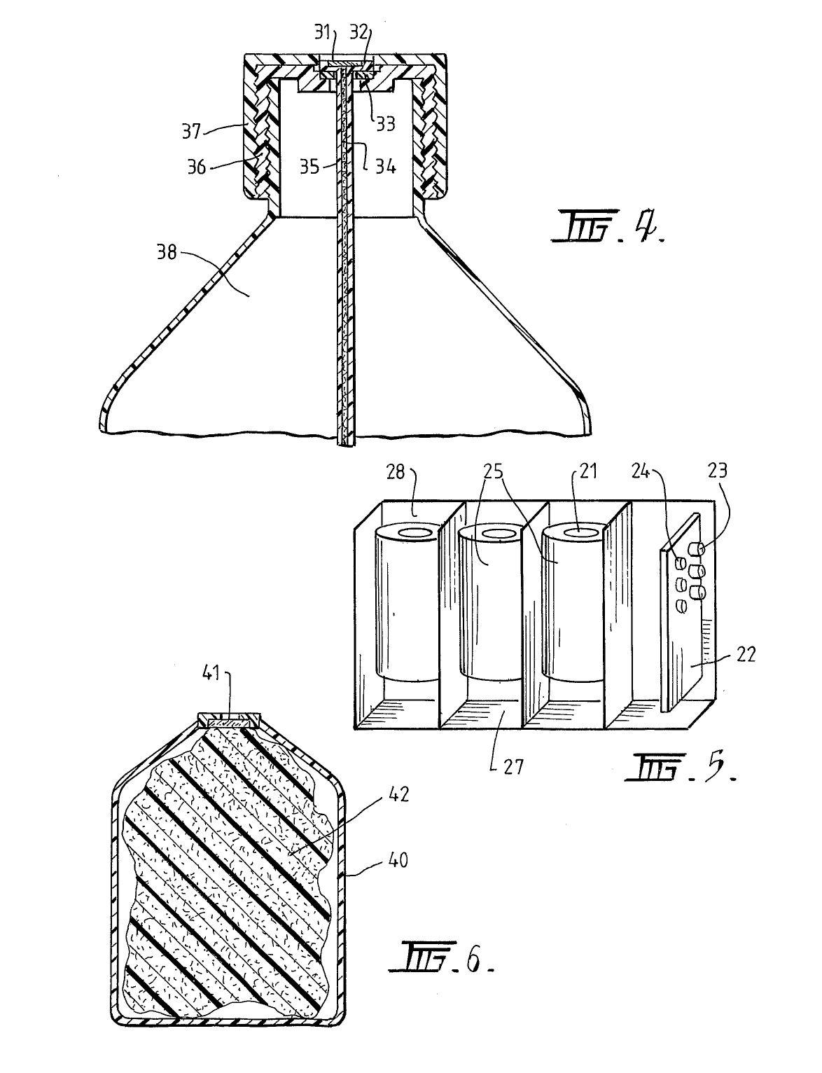

It is also envisaged in the invention as previously stated that a multiple scenting device be provided. A schematic diagram of this is show in FIG. 5 where reservoir units 25 having diffuser electronics 21 are located in a housing 27 having LED displays 23 and controls 24 on panel 22.

A further embodiment of an integrated dispenser is shown in FIG. 6 where a reservoir 40 holds a sponge type material 42 soaked in the scented oil which sponge communicates with a piezo element 41 which acts to disperse the oil.

This latter embodiment is particularly effective while being very simple.

As previously stated the invention is not restricted in terms of dispenser used however it is preferred that the system of the invention be used with a piezo electric actuated dispenser.

Clearly many embodiments of the invention lie within its scope and it is whilst we have described here particular embodiments of the invention it is envisaged that other embodiments of the invention will exhibit any number of and any combination of the features previously described. It is further to be understood that variations and modifications in this can be made without departing from the spirit and scope of the invention.

1-23. (canceled)

24. An apparatus for dispensing aerosols, comprising:
a plurality of aerosol dispensers with each aerosol dispenser of said plurality of aerosol dispensers having a reservoir of a liquid for dispersion; and,
a single control unit; and,
means for actuating said plurality of aerosol dispensers being controlled by said single control unit.

25. The apparatus for dispensing aerosols according to claim 24, wherein said plurality of aerosol dispensers are distributed remotely from one another.

26. The apparatus for dispensing aerosols according to claim 24, wherein said single control unit includes radio transmission means for actuating and controlling said plurality of aerosol dispensers.

27. The apparatus for dispensing aerosols according to claim 24, wherein said single control unit includes wireless transmission means with a hopping or re-routing facility for communicating with distant dispensers.

28. The apparatus for dispensing aerosols according to claim 24, wherein said single control unit includes means for monitoring sensory feedback from the environment of at least one said aerosol dispenser of said plurality of aerosol dispensers and adjusting parameters in response to sensory feedback received.

29. The apparatus for dispensing aerosols according to claim 28, wherein said single control unit includes means for monitoring status of each said aerosol dispenser.

30. The apparatus for dispensing aerosols according to claim 29, wherein said single control unit includes means for signaling need for replenishment of liquid.

31. The apparatus for dispensing aerosols according to claim 24, wherein said single control unit includes means for analyzing effectiveness of aerosol diffusion as would be perceived by a human being.

32. The apparatus for dispensing aerosols according to claim 24, wherein each said aerosol dispenser is integrated and sealed with said reservoir for forming a cartridge for making a replaceable unit.

33. The apparatus for dispensing aerosols according to claim 24, wherein each said aerosol dispenser is a piezo-electrically actuated dispenser.

34. The apparatus for dispensing aerosols according to claim 24, wherein said reservoir includes a sponge therein and liquid flow from said reservoir to a respective said each aerosol dispenser is via said sponge in said reservoir.
